(19) United States
(12) Patent Application Publication
(10) Pub. No.: US 2004/0023236 A1
(43) Pub. Date: Feb. 5, 2004

(54) Chemical and biological hazard sensor system and methods thereof

(76) Inventors: Michael D. Potter, Churchville, NY (US); David R. Chafin, Rochester, NY (US); Dennis Michael Connolly, Rochester, NY (US)

Correspondence Address: Gunnar G. Leinberg, Esq., NIXON PEABODY LLP, Clinton Square, P.O. Box 31051, Rochester, NY, 14603-1051, US

(21) Appl. No.: 10/282,277

(22) Filed: Oct. 28, 2002

Related U.S. Application Data
(60) Provisional application No. 60/336,785, filed on Oct. 26, 2001.

(51) Int. Cl. C12Q 1/68; C12M 1/34

(52) U.S. Cl. 435/6; 435/287.2; 205/777.5

(57) ABSTRACT

The present invention relates to a sensor system containing a member with a stored electrical charge and probe molecules attached to at least a portion of the member. The sensor system also contains at least one common electrode, an input electrode, and an output electrode, where the common electrode and the input and output electrodes are spaced from and on substantially opposing sides of the member from each other and are at least partially in alignment with each other. The member is movable with respect to the common electrode and the input and output electrodes.

The present invention claims the benefit of U.S. Provisional Patent Application Serial No. 60/336,785, filed Oct. 26, 2001, which is hereby incorporated by reference in its entirety.

### FIELD OF THE INVENTION

This invention relates generally to sensors and, more particularly, to a chemical and biological hazard sensor system and methods thereof.

### BACKGROUND OF THE INVENTION

Biological materials, such as proteins, nucleic acid molecules such as DNA and RNA, or whole cells, have become of increasing interest as analytes for clinical tests or detection of hazardous substances. Powerful new molecular biology techniques enable one to assay for congenital or infectious diseases. These same technologies can characterize biological materials for detection of biowarfare agents. Some of these techniques are DNA fingerprinting, restriction fragment length polymorphism (RFLP) analysis, and western and southern blotting. Nucleic acid testing has been made possible due to powerful amplification methods. One can take small amounts of nucleic acids, which would normally be undetectable, and increase or amplify to a degree where useful amounts are present for detection. Protein detection has mainly focused around capture of target molecules by antibody binding and fluorescence detection. Likewise, capture of whole cells for analysis most commonly involves using capture antibodies produced against unique cellular coat proteins that reside on the outside of most cells.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification, and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics, 4:560 (1989) and the ligase chain reaction described in European Patent No. 0320308B1 to Backman et al. Transcription-based amplification methods are described in detail in U.S. Pat. No. 5,766,849 to McDonough et al., U.S. Pat. No. 5,654,142 to Kievits et al., Kwoh et al., Proc. Natl. Acad. Sci. U.S.A., 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol., 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve, 21(8):1064 (1998); Wiedbrauk et al., Journal of Clinical Microbiology, 33(10):2643-6 (1995); Deneer and Knight, Clinical Chemistry, 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology 39(2):485-493 (2001); Al-Soud et al., Journal of Clinical Microbiology, 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092 to Fodor et al., and PCT Publication No. WO 90/15070 to Fodor et al. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection for the presence of specific DNA sequences. Most technologies related to array chips involve the coupling of a probe of known sequence to a substrate that can either be structural or conductive in nature. Structural types of array chips usually involve providing a platform where probe molecules can be constructed base by base or covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location.

However, this method requires the use of fluorescent or radioactive labels as additional materials. Such a system is expensive to use and is not amenable to being made portable for biological sample detection and identification. Furthermore, the hybridization reactions take up to two hours, which for many uses, such as detecting biological warfare agents, is simply too long. Therefore, a need exists for a system which can rapidly detect biological material in samples.

Many techniques are being investigated in order to find a fast and reliable way to identify hazardous substances such as biological pathogens or toxic gases in the environment. One technique used to identify a biological pathogen is to extract the nucleic acid, use enzymes to specifically cut fragments of probe molecules, amplify if necessary, and then hybridize with a specific complement that also includes a tag. The tag may be either a fluorescent substance or a radioactive substance. Measuring the presence or absence of the tag completes the analysis, i.e. luminescence or scintillation. Other techniques include attaching target samples to magnetic beads through hybridization to probe molecules that are previously attached to the beads. Unbound beads are washed away and the presence or absence of magnetism is a measure of the presence or absence of the target pathogen. Toxic gases may be detected through binding to a probe molecule previously attached to a sensor such as a piezoelectric crystal, where a change in resonant frequency indicates a positive event.

Currently, there are no effective biological detectors sensitive enough to detect the presence of only a few molecules within a biological sample. Furthermore, it would be advantageous to develop a highly sensitive sensor that would detect not only nucleic acid molecules but also proteins, whole cells and other molecules.

The present invention is directed to achieving these objectives.

### SUMMARY OF THE INVENTION

The present invention relates to a sensor system containing a member with a stored electrical charge and probe molecules attached to at least a portion of the member. The sensor system also contains at least one common electrode, an input electrode, and an output electrode, where the common electrode and the input and output electrodes are spaced from and on substantially opposing sides of the member from each other and are at least partially in alignment with each other. The member is movable with respect to the common electrode and the input and output electrodes.

The present invention also relates to a method for making a sensor. The method first involves providing a member with a stored electrical charge. Then, probe molecules attached to at least a portion of the member are provided. Finally, at least one common electrode, an input electrode, an output electrode are provided, where the common electrode and the input and output electrodes are spaced from and on substantially opposing sides of the member from each other and are at least partially in alignment with each other. The member is movable with respect to the common electrode and the input and output electrodes.

Another aspect of the present invention relates to a method for detecting a target material in a sample. The method first involves exposing probe molecules attached to at least a portion of a member with stored electrical charge to a sample potentially containing the target material. Then, a resonant frequency of the member in response to the exposure is monitored and the monitored resonant frequency is outputted.

The present invention provides a highly sensitive sensor system and method for detecting target biological and chemical materials such as nucleic acids, proteins, whole cells from relatively crude sample preparations, or toxic gases. The target material is captured by a probe molecule anchored to metal, glass or other compatible surfaces in the sensor, where the binding of target molecules or cells triggers a resonant frequency change within the device. Since the frequency of the detector is dependent on the mass, and the resonator beam or diaphragm can be made very small, binding of only a few molecules or cells can be detected. The present invention utilizes the strong output signal achieved by exploiting the phenomenon of embedded electronic charge. Additionally, the present invention can be made very small and can be easily integrated with standard semiconductor devices such as a CMOS circuit.

### BRIEF DESCRIPTION OF DRAWINGS

FIG. 1 is a side, cross-sectional view of a sensor system with a member configured as a cantilever beam in accordance with one embodiment of the present invention.

FIG. 2 is a side, cross-sectional view of a sensor system with a member configured as a double fixed beam in accordance with another embodiment of the present invention.

FIGS. 3-12 illustrate the sequence of steps necessary for fabricating a sensor for detecting hazardous substances in accordance with one embodiment of the present invention.

FIG. 13 is a graph that depicts the flatband voltage before and after charge injection using an aluminum top electrode—composite insulator—semiconductor (MOS) capacitor. The change in flatband voltage is used to calculate the stored charge density.

FIG. 14 is a graph that shows post charge injection flatband voltage as a function of log time in minutes.

### DETAILED DESCRIPTION

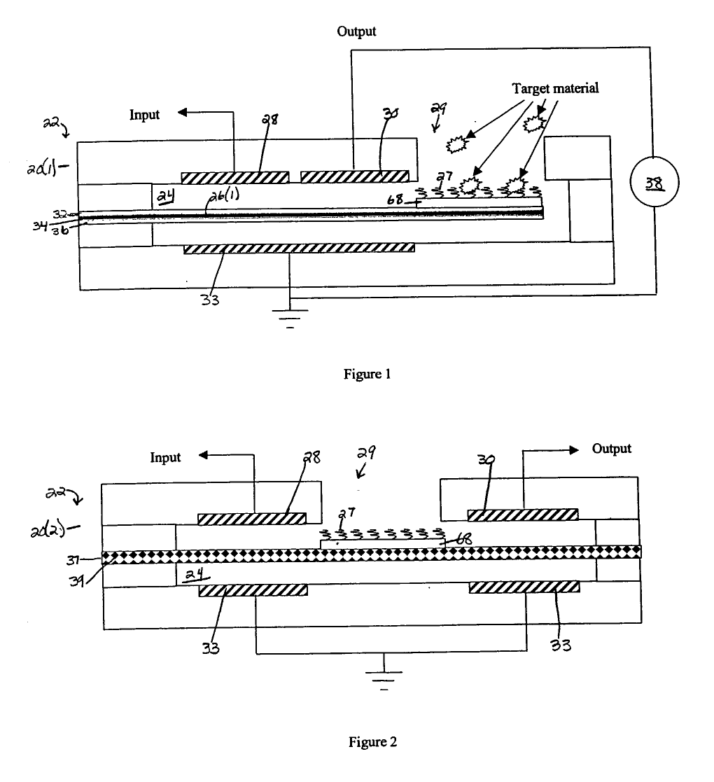

A sensor system 20(1) for detecting environmental hazards in accordance with one embodiment of the present invention is illustrated in FIG. 1. In this particular embodiment, the sensor system 20(1) includes a housing 22 with a chamber 24, a member 26(1) with a stored electrical charge, probe molecules 27, and an input electrode 28, an output electrode 30, and a common electrode 33, although sensor system 20(1) may comprise other arrangements of components, such as having two or more common electrodes. The present invention provides a simpler and more effective system and method for detecting environmental hazards.

Referring more specifically to FIG. 1, the housing 22 has an internal chamber 24 and is made of a variety of layers, although other types of structures in other configurations and with other numbers of layers, such as one or more, made of other materials can be used. The size of the housing 22 and of the chamber 24 can also vary as required by the particular application. The chamber 24 includes an opening 29 to allow a sample to be introduced, although the chamber can have other numbers of openings.

The member 26(1) may have one end or edge of the member 26(1) connected to the housing 22 and have an opposing end or edge which is free and spaced from an inner wall of the housing 22 to form a cantilever beam as shown in FIG. 1, although other arrangements can be used. For example, the member 26(1) can extend across the chamber and may have both ends fixed to the housing 22 as shown in FIG. 2. Alternatively, the member can extend across the chamber and be connected along all of its edges to the housing to form a diaphragm.

The member 26(1) can store embedded electrical charge. The member 26(1) has a pair of layers 32 and 36 of dielectric material, such as silicon oxide, silicon dioxide, silicon nitride, aluminum oxide, tantalum oxide, tantalum pentoxide, titanium oxide, titanium dioxide, barium strontium titanium oxide, although other types of materials which can hold a electrical charge and other numbers of layers, such as a member with one layer or three or more layers can be used. The layers 32 and 36 are seated against each other along an interface 34 where the electrical charge is stored. The member 26(1) can hold an electrical charge on the order of at least 1×10¹⁰ charges/cm².

Probe molecules 27 are attached to the desired area of the member 26(1). The area of the member 26(1) where the probe molecules will be attached may be coated with a layer of material 68 such as gold to enhance the attachment of the probe molecules.

The electrodes 28, 30, 33 are located in the inner walls of the housing 22 in chamber 24, although other configurations for connecting the electrodes 28, 30, 33 to the housing 22 can be used, such as having each of the electrodes 28, 30, 33 located in the inner wall of the housing 22 and spaced from the chamber 24 by one or more layers of insulating material, or by having each of the electrodes 28, 30, 33 seated on the inner walls of the housing 22 in the chamber 24. The input and output electrodes 28, 30 are in substantial alignment with the common electrode 33 and are spaced from and located on substantially opposing sides of the member 26(1), although other configurations can be used.

The input and output electrodes 28, 30 and the common electrode 33 are initially spaced substantially the same distance from the member 26(1), although other configurations can be used. The spacing between each of the electrodes 28, 30, 33 and the member 26(1) depends on the permittivity of the material(s) and/or fluid(s) in the chamber 24 between each of the electrodes 28, 30, 33 and the member 26(1) and the desired initial potential difference. By way of example only, in this particular embodiment the distance between each of the electrodes 28, 30, 33 and the member 26(1) is about 1.0 micron and the initial potential difference is zero.

A resonator monitoring system 38 is coupled to the pair of input/output electrodes 28, 30 and the common electrode 33, although other types of devices can be coupled to the electrodes 28, 30, 33. The resonator monitoring system 38 is able to monitor, measure, and output the resonant frequency of the member 26(1) before and after the sample is introduced, although the resonator monitoring system 38 may have other functions. For example, based on the determined resonant frequency after the sample is introduced, the resonator monitoring system 38 may be programmed with instructions stored in a memory and executed by a processor to determine what substance or substances may be in the sample and whether they pose a hazard. By way of example only, the measured resonant frequency after the sample is introduced may simply be compared against a look up table of resonant frequencies which are each correlated to different quantities of substances. Based on the closest match, the resonator monitoring system 38 will output a result of how much of the target substance is present in the sample.

Referring to FIG. 2, sensor system 20(2) in accordance with another embodiment is shown. Elements in FIG. 2 which are like elements shown and described in FIG. 1 will have like numbers and will not be shown and described in detail again here. In this particular embodiment, the member 26(2) extends across the chamber and has both ends fixed to the housing 22, and the probe molecules 27 are attached to the center of the member, although the probe molecules can be attached wherever desired along the length of the member. In this particular embodiment, the member 26(2) stores embedded electrical charge 39 in a single layer 37 of dielectric material, such as silicon oxide, silicon dioxide, silicon nitride, aluminum oxide, tantalum oxide, tantalum pentoxide, titanium oxide, titanium dioxide, barium strontium titanium oxide, although other types of materials which can hold a electrical charge.

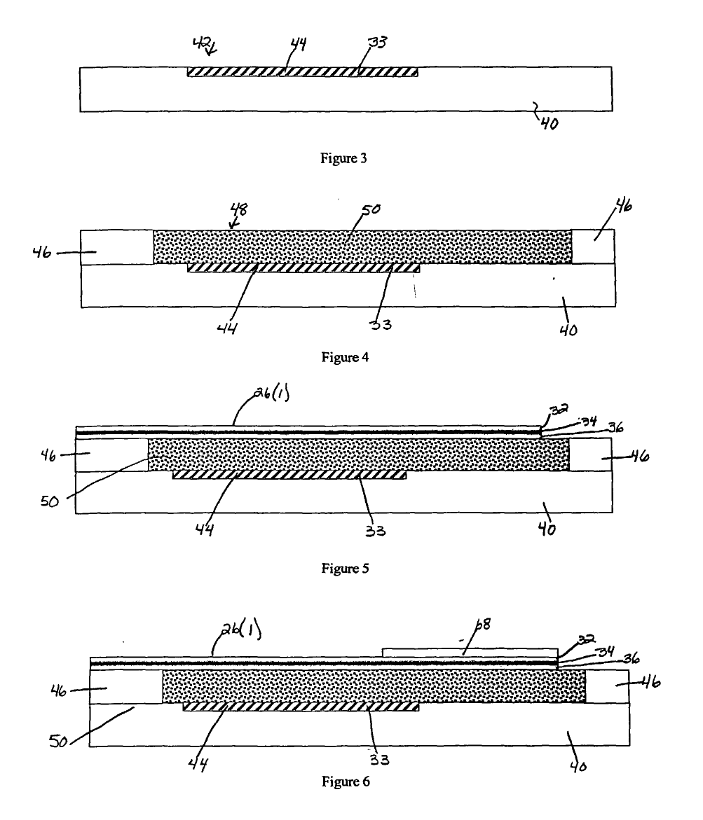

A method for making a sensor system 20(1) in accordance with one embodiment of the present invention is described below with reference to FIGS. 3-12. To make a sensor system 20(1), a suitable substrate 40, such as silicon oxide on silicon, is provided as shown in FIG. 3, although other types of materials could be used. A first trench 42 is formed in the substrate 40, using standard fabrication means, and the first trench 42 is filled with a first conductive layer 44, such as aluminum, although other types of materials could be used. The first conductive layer 44 may be planarized so that only the first trench 42 is filled with the first conductive layer 44. By way of example only, this may be done by standard chemical mechanical planarization (CMP) processing, although other techniques can be used. The resulting first conductive layer 44 in the first trench 42 forms the common electrode 33. The first conductive layer 44 could be etched to form two separate common electrodes. Alternatively, the common electrode could be a conductive substrate.

Referring to FIG. 4, a first insulating layer 46, such as silicon dioxide, is deposited on the first conductive layer 44 and a portion of the substrate 40, although other types of materials could be used. A second trench 48 is formed in the first insulating layer 46 which is at least in partial alignment with the common electrode 33. The second trench 48 is etched to the surface of the common electrode 33, although other configurations can be used, such as leaving a portion of the first insulating layer 46 over the common electrode 33. The second trench 48 is filled with a first sacrificial layer 50, such as poly silicon, and may be planarized, although other types of materials could be used for first sacrificial layer 50. By way of example, the planarizing of the first sacrificial layer 50 may be done by standard CMP processing, although other techniques can be used.

Referring to FIG. 5, a member 26(1) which can store an electrical charge, such as a fixed or floating electrical charge, is deposited on a portion of the first insulating layer 46 and the first sacrificial material 50 so that the member 26(1) is spaced from one portion of the first insulating layer 46, although other arrangements can be used. In this particular embodiment, the member 26(1) comprises two layers 32 and 36 of insulating material, such as silicon oxide and silicon nitride, silicon oxide and aluminum oxide, or any other combination of materials that can store fixed electrical charge can be deposited as the member 26(1). Additionally, the member 26(1) may comprise other numbers of layers of material, such as a member with a single layer or multiple layers. For example, a tri-layer of silicon oxide—silicon nitride—silicon oxide may be used.

The member 26(1) can move towards and away from the common electrode 33 and the input and output electrodes 28, 30.

Electrical charge is injected into a portion of the member 26(1), where probe molecules 27 are not attached, although other arrangements can be used. A variety of techniques for injecting electrical charge can be used, such as a low to medium energy ballistic electron source or by utilizing a sacrificial conductive layer (not shown) disposed on top of the member 26(1) and subsequently applying an electric field sufficient to inject electrons into the member 26(1).

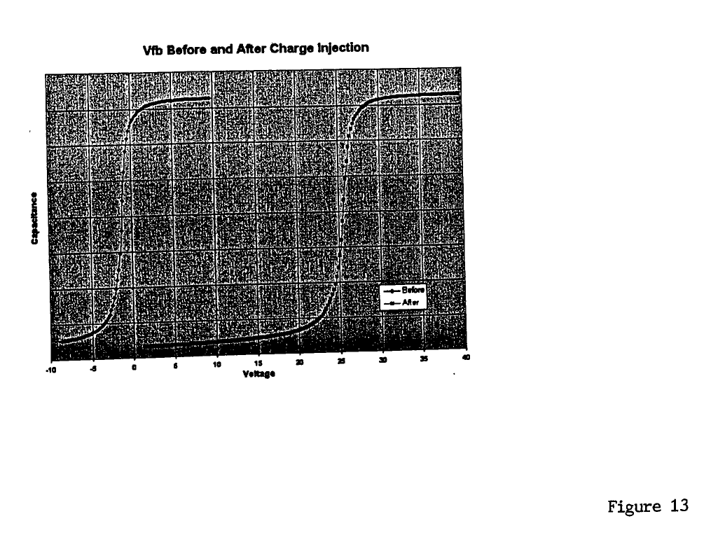

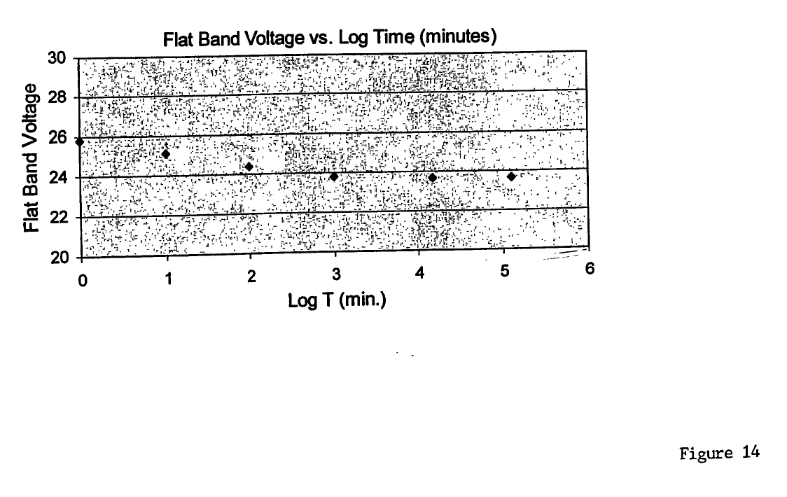

By way of example only, a test structure using a lightly doped n-type semiconductor wafer for the common electrode 33 and aluminum for the input/output electrodes 28, 30 was fabricated in order to measure the magnitude and retention time of the embedded electrical charge. FIG. 13 shows the flatband voltage before and after charge injection using the aluminum electrode—composite insulator—semiconductor (MOS) capacitor. The change in flatband voltage was used to calculate the stored electrical charge densities before and after high field electron charge injection. As indicated in FIG. 14, post electron injection results showed a stored electrical charge density of 1×10¹³ electrons per cm² with a retention time of many years.

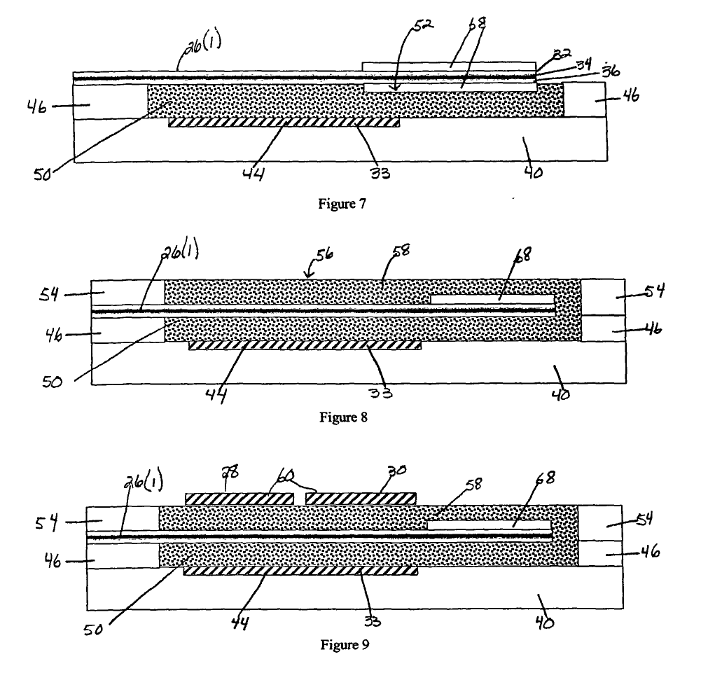

Referring to FIG. 6, a layer of material 68 such as gold may be deposited on a portion of the member 26(1) where the probe molecules will be attached, in order to enhance the attachment of the probe molecules. The layer of material 68 can also be deposited on portions of both sides of the member 26(1) as shown in FIG. 7. In this particular embodiment, a third trench 52 is formed in the first sacrificial material 50 and another layer of material 68 is deposited and planarized before the member 26(1) is deposited on a portion of the first insulating layer 46 and the first sacrificial material 50. In this particular embodiment, it is not necessary to inject electrical charge into only the portion of the member where probe molecules are not attached, because the layer of material may act as a shield.

Referring to FIG. 8, a second insulating layer 54, such as silicon dioxide is deposited on the member 26(1) and a portion of the first insulating layer 46, although other types of materials can be used. Next, a fourth trench 56 is etched in the second insulating layer 54 to the member 26(1), although the fourth trench 56 can be etched to other depths. The fourth trench 56 is in substantial alignment with the second trench 48, although other arrangements can be used as long as the fourth trench 56 is at least in partial alignment with the second trench 48. The fourth trench 56 is filled with a second sacrificial material 58, such as poly silicon, although other types of material can be used. The second sacrificial material 58 may be planarized.

Referring to FIG. 9, a second conductive layer 60, such as aluminum, is deposited on at least a portion of the second insulating layer 54 and the second sacrificial material 58, although other types of materials can be used. The second conductive layer 60 is etched to form an input electrode 28 and an output electrode 30 in this embodiment.

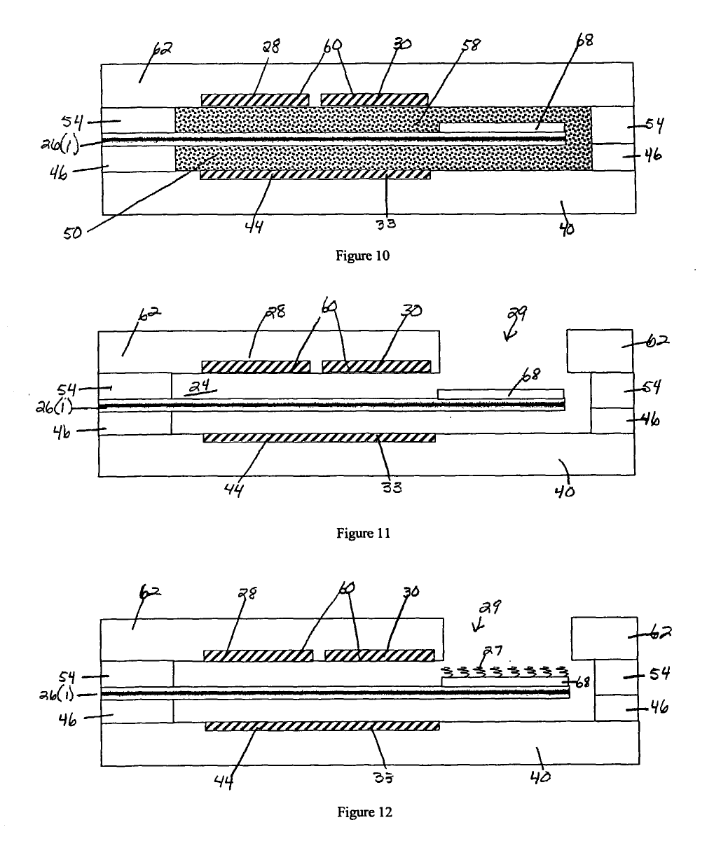

Referring to FIG. 10, a third insulating layer 62, such as silicon dioxide, is deposited over at least a portion of the second insulating layer 54 and the input and output electrodes 28, 30 to encapsulate the input and output electrodes 28, 30, although other types of materials can be used.

Referring to FIG. 11, holes (not shown) to the electrodes 28, 30, 33 are formed to provide contact points for electrical coupling and one or more openings or vias 29 are also etched to provide access to the first and second sacrificial layers 50 and 58. The first and second sacrificial materials 50, 58 are removed through the openings to provide an access point to the chamber for introducing a sample. A variety of techniques can be used to remove the sacrificial materials 50, 58. For example, if the sacrificial material is poly silicon, the etchant may be xenon difluoride. In the particular embodiment where the member is connected to the housing to form a diaphragm, removing the sacrificial material forms a compartment in chamber 24 which can be filled with a gas to act as a damping means or be a vacuum.

Referring to FIG. 12, probe molecules 27 are attached to the desired area of the member 26(1). The area of the member 26(1) where the probe molecules will be attached may be coated with a layer of material 68, such as gold, to enhance the attachment of the probe molecules. Details on methods of attaching biological molecules to electrically conductive surfaces can be found in U.S. patent application Ser. No. 10/159,429, filed on May 30, 2002, which is hereby incorporated by reference in its entirety.

In one embodiment of the invention, the probe molecules 27 can be antibodies specific for a protein within a sample of interest. The protein may reside in the interior of the cell or on the exterior of the cell. In the case of a protein residing on the interior of the cell, the cells will be disrupted prior to detection. Furthermore, some cellular debris may be removed prior to capture of molecules of interest. Alternatively, the protein may reside on the exterior of the cell, in which case no disruption of the cell may be necessary. In this embodiment, whole cells may be captured, thereby causing a rather large change in the mass and, therefore, the resonant frequency of the device and a rather large signal. In another embodiment of the invention, the probe molecules 27 are molecules capable of binding to toxic gases.

In another embodiment of the invention, the probe molecules 27 can be DNA, RNA, or oligonucleotide molecules. The oligonucleotide probes can be in the form of DNA, RNA, or chemically modified nucleic acid molecules or oligonucleotide analogues. An “oligonucleotide analogue” refers to a polymer with two or more monomeric subunits, wherein the subunits have some structural features in common with a naturally occurring oligonucleotide which allow it to hybridize with a naturally occurring nucleic acid in solution. For instance, structural groups are optionally added to the ribose or base of a nucleoside for incorporation into an oligonucleotide, such as a methyl or allyl group at the 2′-O position on the ribose, or a fluoro group which substitutes for the 2′-O group, or a bromo group on the ribonucleoside base. The phosphodiester linkage, or “sugarphosphate backbone” of the oligonucleotide analogue is substituted or modified, for instance with methyl phosphonates or O-methyl phosphates. Another example of an oligonucleotide analogue includes “peptide nucleic acids” in which native or modified nucleic acid bases are attached to a polyamide backbone. Oligonucleotide analogues optionally comprise a mixture of naturally occurring nucleotides and nucleotide analogues. Oligonucleotide analogue arrays composed of oligonucleotide analogues are resistant to hydrolysis or degradation by nuclease enzymes such as RNAase A. This has the advantage of providing the array with greater longevity by rendering it resistant to enzymatic degradation. For example, analogues comprising 2′-O-methyloligoribonucleotides are resistant to RNAase A.

Many modified nucleosides, nucleotides, and various bases suitable for incorporation into nucleosides are commercially available from a variety of manufacturers, including the SIGMA chemical company (Saint Louis, Mo.), R&D systems (Minneapolis, Minn.), Pharmacia LKB Biotechnology Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill. Methods of attaching bases to sugar moieties to form nucleosides are known. See, e.g., Lukevics and Zablocka, Nucleoside Synthesis: Organosilicon Methods Ellis Horwood Limited Chichester, West Sussex, England (1991), which is hereby incorporated by reference in its entirety. Methods of phosphorylating nucleosides to form nucleotides, and of incorporating nucleotides into oligonucleotides are also known. See, e.g., Agrawal (ed), Protocols for Oligonucleotides and Analogues, Synthesis and Properties, Methods in Molecular Biology, volume 20, Humana Press, Towota, N.J. (1993), which is hereby incorporated by reference in its entirety.

Methods of synthesizing desired oligonucleotide probes are known to those of skill in the art. In particular, methods of synthesizing oligonucleotides and oligonucleotide analogues can be found in, for example, Oligonucleotide Synthesis: A Practical Approach, Gait, ed., IRI Press, Oxford (1984); Kuijpers, Nucleic Acids Research 18(17):5197 (1994); Dueholm, J. Org. Chem., 59:5767-5773 (1994); and Agrawal (ed.), Methods in Molecular Biology, 20, which are hereby incorporated by reference in their entirety. Shorter oligonucleotide probes have lower specificity for a target nucleic acid molecule, that is, there may exist in nature more than one target nucleic acid molecule with a sequence of nucleotides complementary to the oligonucleotide probe. On the other hand, longer oligonucleotide probes have decreasingly smaller probabilities of containing complementary sequences to more than one natural target nucleic acid molecule. In addition, longer oligonucleotide probes exhibit longer hybridization times than shorter oligonucleotide probes. Since analysis time is a factor in a commercial device, the shortest possible probe that is sufficiently specific to the target nucleic acid molecule is desirable.

In one embodiment of the present invention, a portion of the member 26(1) or 26(2) is coated with a metal. Different kinds of metals require different kinds of attachment chemistries to attach the probe molecules. In other embodiments of the present invention, any material that can be fabricated on the surface of the member 26(1) or 26(2) can be used, provided that there is a way of attaching the probe molecules.

Referring to FIG. 1, the resulting sensor system 20(1) is shown. A resonator monitoring system 38 may be coupled to the output electrode 30 and the common electrode 33, although other types of devices could be coupled to the output electrode 30 and the common electrode 33.

The method for making the sensor system 20(2) shown in FIG. 2 is the same as the method described for making the sensor system 20(1) as described with reference to FIGS. 3-12, except, in this particular embodiment, the member 26(1) is deposited in a manner so that the member 26(1) extends across the chamber and has both ends fixed to the housing 22.

The operation of the sensor system 20(1) in accordance with one embodiment will be described with reference to FIG. 1. A changing potential of a drive signal applied to electrodes 28 and 33 causes the member 26(1) to oscillate due to the force imparted on the member 26(1) by the electric field (F=QE). The output signal is maximum when the input drive signal frequency is the same as the resonant frequency of the member 26(1). The resonant frequency of member 26(1) is dependent upon geometry and materials properties. Since the effect of mass will most likely increase faster than the effect of any additional materials properties (e.g. a change in the effective Young's Modulus), the resonant frequency is likely to decrease.

When the sensor system 20(1) is exposed to a sample that contains a target material, the target material will bind to the probe molecules, which will cause a change in the resonant frequency of the member 26(1). Detection of a binding event is accomplished by measuring the resonant frequency of the member 26(1), which can be done by measuring a resonant frequency shift with resonator monitoring system 38 by sweeping the input frequencies or by noting an output amplitude change when driving the input at the original resonant frequency. The resonator monitoring system 38 can also perform a variety of other functions such as outputting the changed resonant frequency or determining the quantity of target material present in the sample based on the measurements.

The operation of the sensor system 20(2) shown in FIG. 2 is the same as that for the sensor system 20(1), except that the resonant frequency of member 26(2) will be different from the resonant frequency of member 26(1).

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed:

1. A sensor system comprising:
a member with a stored electrical charge;
probe molecules attached to at least a portion of the member; and
at least one common electrode, an input electrode, and an output electrode, the common electrode and the input and output electrodes being spaced from and on substantially opposing sides of the member from each other and are at least partially in alignment with each other, wherein the member is movable with respect to the common electrode and the input and output electrodes.

2. The system as set forth in claim 1 further comprising a resonator monitoring system coupled to the common electrode and the input and output electrodes.

3. The system as set forth in claim 1 further comprising a housing with a chamber, wherein the member is connected to the housing and extends at least partially across the chamber and the electrodes are connected to the housing.

4. The system as set forth in claim 3 wherein the member is connected to the housing to form a cantilever beam.

5. The system as set forth in claim 3 wherein the member is connected to the housing to have both of its ends fixed to the housing.

6. The system as set forth in claim 3 wherein the member extends across the chamber and is connected to the housing to form a diaphragm.

7. The system as set forth in claim 1 wherein the at least one common electrode comprises two separate electrodes.

8. The system as set forth in claim 1 wherein the member comprises two or more dielectric layers.

9. The system as set forth in claim 1 wherein the member comprises a single dielectric layer.

10. The system as set forth in claim 1 wherein the member is made from one or more materials selected from a group consisting of silicon oxide, silicon dioxide, silicon nitride, aluminum oxide, tantalum oxide, tantalum pentoxide, titanium oxide, titanium dioxide, barium strontium titanium oxide.

11. The system as set forth in claim 1 further comprising a layer of material deposited on at least a portion of the member, wherein the probe molecules are attached to the layer of material.

12. The system as set forth in claim 11 wherein the layer of material is deposited on at least a portion of both sides of the member.

13. The system as set forth in claim 1 wherein the probe molecules are oligonucleotide probes.

14. The system as set forth in claim 1 wherein the probe molecules are proteins or antibodies.

15. The system as set forth in claim 1 wherein the probe molecules are capable of binding to toxic gases.

16. A method for making a sensor, the method comprising:
providing a member with a stored electrical charge;
providing probe molecules attached to at least a portion of the member; and
providing at least one common electrode, an input electrode, an output electrode, the common electrode and the input and output electrodes being spaced from and on substantially opposing sides of the member from each other and are at least partially in alignment with each other, wherein the member is movable with respect to the common electrode and the input and output electrodes.

17. The method as set forth in claim 16 further comprising providing a resonator monitoring system coupled to the common electrode and the output electrode.

18. The method as set forth in claim 16 further comprising providing a housing with a chamber, wherein the member is connected to the housing and extends at least partially across the chamber and the electrodes are connected to the housing.

19. The method as set forth in claim 18 wherein the member is connected to the housing to form a cantilever beam.

20. The method as set forth in claim 18 wherein the member is connected to the housing to have both of its ends fixed to the housing.

21. The method as set forth in claim 18 wherein the member extends across the chamber and is connected to the housing to form a diaphragm.

22. The method as set forth in claim 16 wherein the at least one common electrode comprises two separate electrodes.

23. The method as set forth in claim 16 wherein the member comprises two or more dielectric layers.

24. The method as set forth in claim 16 wherein the member comprises a single dielectric layer.

25. The method as set forth in claim 16 wherein the member is made from one or more materials selected from a group consisting of silicon oxide, silicon dioxide, silicon nitride, aluminum oxide, tantalum oxide, tantalum pentoxide, titanium oxide, titanium dioxide, barium strontium titanium oxide.

26. The method as set forth in claim 16 further comprising a layer of material deposited on at least a portion of the member, wherein the probe molecules are attached to the layer of material.

27. The method as set forth in claim 26 wherein the layer of material is deposited on at least a portion of both sides of the member.

28. The method as set forth in claim 16 wherein the probe molecules are oligonucleotide probes.

29. The method as set forth in claim 16 wherein the probe molecules are proteins or antibodies.

30. The method as set forth in claim 16, wherein the probe molecules are capable of binding to toxic gases.

31. A method for detecting a target material in a sample, the method comprising:
exposing probe molecules attached to at least a portion of a member with stored electrical charge to a sample potentially containing the target material;
monitoring a resonant frequency of the member in response to the exposure;
outputting the monitored resonant frequency.

32. The method as set forth in claim 31 further comprising determining the presence of the target material based on the monitored resonant frequency.

33. The method as set forth in claim 31 further comprising a layer of material deposited on at least a portion of the member, wherein the probe molecules are attached to the layer of material.

34. The method as set forth in claim 33 wherein the layer of material is deposited on at least a portion of both sides of the member.

35. The method as set forth in claim 31 wherein the probe molecules are oligonucleotide probes.

36. The method as set forth in claim 31 wherein the probe molecules are proteins or antibodies.

37. The method as set forth in claim 31 wherein the probe molecules are capable of binding to toxic gases.
